(19) United States
(12) Patent Application Publication
(10) Pub. No.: US 2016/0271285 A1
(43) Pub. Date: Sep. 22, 2016

(54) PROCESSES FOR SANITIZING AND DECONTAMINATING CRUMB RUBBER OR SYNTHETIC TURF

(71) Applicant: Synthetic Turf Treat, LLC, Sarasota, FL (US)

(72) Inventors: Stanley Reed Weller, Marietta, GA (US); Suhail Massad, Marietta, GA (US); Vincent Scuilla, Sarasota, FL (US); Lauren G. Danielson, Bradenton, FL (US)

(73) Assignee: Synthetic Turf Treat, LLC, Sarasota, FL (US)

(21) Appl. No.: 15/071,769

(22) Filed: Mar. 16, 2016

Related U.S. Application Data
(60) Provisional application No. 62/135,828, filed on Mar. 20, 2015.

(51) Int. Cl. A61L 9/01; A61L 2/10; A61L 2/20; A61L 2/18

(52) U.S. Cl. CPC A61L 9/01; A61L 2/186; A61L 2/18; A61L 2/10; A61L 2/202; A61L 2/20; A61L 2209/212; A61L 2209/211

(57) ABSTRACT

A process and kit for decontaminating crumb rubber or synthetic turf involves use of an antimicrobial agent for killing microorganisms embedded within or upon the crumb rubber or the synthetic turf, and/or subsequently using a non-pathogenic microbe to degrade organic materials in the crumb rubber or synthetic turf which degrades organic materials which may harbor odor causing organisms therefore preventing them from becoming established.

### CROSS-REFERENCE TO RELATED APPLICATIONS

This application claims the benefit of U.S. Provisional Application No. 62/135,828, filed Mar. 20, 2015, which is incorporated herein by reference in its entirety.

### FIELD OF THE DISCLOSURE

This disclosure relates to decontamination and/or sanitization of surfaces and more particularly to decontamination and/or sanitization of synthetic turf and crumb rubber.

### BACKGROUND OF THE DISCLOSURE

Synthetic turf is a cover that is made to look and feel like natural grass which can be installed either indoors or outdoors. The simulated blades of grass of synthetic turf are typically comprised of a synthetic polymer, such as polyethylene, polypropylene or nylon.

Synthetic turf has become increasingly popular since the 1990s when sand and rubber infill were introduced to improve player performance and safety. Approximately half of the National Football League playing fields currently use artificial turf. It has also become increasingly popular for baseball, soccer, golf and many other types of sports that were conventionally played on natural grass.

Artificial turf and crumb rubber are also useful for pet and veterinary applications (e.g., kennels and dog runs), day care center applications, and various other retail, commercial and residential property applications (e.g., patios, yards, poolsides, landscapes, etc.).

It was, and in many cases still is, believed that artificial turf requires very little maintenance, other than repairing damaged turf, removing debris, checking and maintaining proper infill levels, and occasionally brushing the turf as needed to preserve the appearance of the artificial turf. However, due to the fact that sports are played on these fields, there are organic substances that come onto the fields and are not easily cleaned. These organic substances include human and animal bodily fluids and/or solids, such as sweat, saliva, urine, feces and blood, as well as spilled drinks and food items that can fall into the turf and become embedded onto both the synthetic turf fibers and the infill. Infill can be made of various substances, such as rubber, sand, silica, zeolite, crumb rubber, rubber coated sand and the like. Organic substances on the infill and the synthetic turf often cause a significant odor issue. Additionally, these degrading organic substances attract deleterious bacteria. Synthetic turf, originally marketed as maintenance free, needs to be maintained to avoid contamination and odor issues.

Crumb rubber is used as a base material for playgrounds, athletic fields in non-turf areas, walking trails, agrimats, animal footing, animal runs, and landscape areas.

### SUMMARY OF THE DISCLOSURE

In certain aspects of this disclosure, an antimicrobial substance is applied to crumb rubber, synthetic turf and/or infill for synthetic turf to reduce viable populations of both pathogenic organisms that are a concern to human or animal health and non-pathogenic organisms that can produce odors during metabolization of organic contaminants.

In certain other aspects of this disclosure, microbes capable of degrading organic matter are applied to crumb rubber or synthetic turf to reduce natural degradation of organic contaminants by odor causing organisms.

In accordance with a further aspect of this disclosure, a comprehensive process for improving and maintaining hygienic synthetic turf or crumb rubber includes a first step of applying an antimicrobial substance to crumb rubber, synthetic turf and/or infill for synthetic turf to reduce viable populations of organisms, and a second step of applying microbes capable of degrading organic contaminants in the crumb rubber or artificial turf.

### DETAILED DESCRIPTION

The antimicrobial treatment is applied to either indoor or outdoor synthetic turf such that it will soak down between one-quarter and three inches, or to the base if longer, into the synthetic turf, thereby coming into contact with odor causing organisms and deleterious bacteria on or in the synthetic turf and/or infill (if present). The indoor non-residual antimicrobial treatments include but are not limited to peroxide chemistry, chlorine dioxide chemistry, steam, ozone or ultraviolet light. Antimicrobials that can be used outdoors include but not limited to bleach, inorganic peroxides, quaternary ammonium compounds or combinations thereof or any of the non-residual antimicrobials listed below for indoor use. The antimicrobials can be used according to directions on a label for that product if it is EPA registered or at an effective rate to provide elimination of odor causing organisms if not EPA registered. The antimicrobial treatment is used to kill off pathogenic and/or odor causing microbes that may be on the infill or synthetic turf. The antimicrobial can be applied in a liquid form (e.g., dissolved in water) and allowed to dry (e.g., approximately 24 hours). Crumb rubber can be similarly treated.

In a microbial treatment, microbes can be applied in a wet form in order to soak down one-quarter to three inches into the infill. Suitable microbes include non-pathogenic spore-based microbes that effectively degrade organic material that would otherwise be susceptible to degradation by odor causing organisms, but which themselves do not produce a substantial odor. This step improves the hygiene of synthetic turf and infill that can be extremely difficult to clean. Spores will remain on the infill and fibers, waiting for additional organic material to appear, and favorable conditions to develop, so that they will then activate to degrade these materials. Thus, as the turf is used, it will provide continuing treatment for odor control and degradation of organic matter.

The microbial treatment can be performed at the time of manufacture or installation of the artificial turf by spraying the infill prior to its application to the synthetic turf to ensure that the infill is pretreated to allow for maximum cleaning and odor control from the start.

The disclosed synthetic turf maintenance treatment can be used on indoor or outdoor synthetic turf, and can include various sporting areas that use synthetic turf, such as golf courses, baseball fields, football fields, soccer fields, indoor practice fields, arenas and schools, as well as retail or residential installations, and on playgrounds, athletic fields, turf areas, walking trails, agrimats, animal footings and runs and landscape areas made of crumb rubber.

The antimicrobial agent can be generally any agent that can safely and effectively kill microorganisms on and embedded within crumb rubber, or synthetic turf and/or within infill material, and which can be effectively neutralized or deactivated to allow a non-pathogenic microbe to be subsequently applied and effectively function to digest or degrade organic materials in the synthetic turf or infill material, including animal or human bodily fluids such as blood, sweat, saliva, urine and feces; food particles; and spilled beverages. The antimicrobial can be one that does not leave a residue after application, or at least does not leave a perceivable residue that degrades the appearance of the artificial turf, and does not inhibit the subsequent effectiveness of the non-pathogenic microbes. Examples of antimicrobial agents that can be employed in certain aspects of this disclosure include hypochlorite solutions, chlorine dioxide gas or solutions, iodophor solutions, and peroxide solutions. In certain other aspects of this disclosure, steam (dry, saturated or superheated), ultraviolet radiation or ozone can be used as an antimicrobial agent. The antimicrobial solution can comprise an effective amount of an antimicrobial agent dissolved in water. Examples of halogen based antimicrobial agents include hypochlorous acid, sodium hypochlorite and calcium hypochlorite or other bromine or iodine based antimicrobials, which may be used at an appropriate and effective concentration. Examples of iodophor antimicrobial agents include povidone-iodine complex, poloxamer-iodine complex, and surfactant-iodine complexes, which can be used at concentrations effective for intended use, Examples of peroxide antimicrobial agents include hydrogen peroxide; peroxy acid (peracetic acid); inorganic peroxides, such as ammonium persulfate and sodium persulfate; and organic peroxide, such as acetyl acetone peroxide and acetyl benzoyl peroxide. A peroxide antimicrobial can be present in the antimicrobial solution at a concentration of about 0.1% to 60% by weight or from 0.1% to 35% by weight. The antimicrobial agent can be supplied in a powder form that is mixed with water prior to application, in a concentrated liquid form that is diluted prior to or during application to crumb rubber, synthetic turf and/or synthetic turf infill, or in a ready-to-use liquid or foam that can be directly applied to synthetic turf, synthetic turf infill, or crumb rubber. The antimicrobial solution or concentrate can be a component (a pail, drum or other container holding a solution, concentrate or dried powder) of a kit for decontaminating synthetic turf or infill material, along with a container (a pail, drum or the like holding a solution, concentrate or dried spores) holding a non-pathogenic microbe that is capable of degrading organic material which may harbor odor-causing organisms, and instructions for their use in accordance with this disclosure. The microbe can be supplied in a powder form that is mixed with water prior to application, in a concentrated liquid form that is diluted prior to or during application to crumb rubber, synthetic turf and/or synthetic turf infill, or in a ready-to-use liquid or foam that can be directly applied to synthetic turf, synthetic turf infill, or crumb rubber.

Because of adverse health effects associated with ozone exposures above about 60 nmol/mol in air or chlorine dioxide exposures above 0.1 ppm, fumigation can be performed in vacated facilities and it is envisioned that a robotic system for a fumigation process could be employed to dispense effective amounts of ozone or chlorine dioxide gas onto and into the artificial turf or crumb rubber to kill microorganisms embedded therein without adverse effects to humans, animals or the environment. For example, a robotic vehicle carrying an ozone generator or chlorine dioxide generator could be programmed to move along a patterned path that exposes a designated area of artificial turf or crumb rubber to ozone or chlorine dioxide antimicrobial treatment. Alternatively, ozone or chlorine dioxide treatments can be applied by an individual that is appropriately protected.

As another alternative, a robotic system or manually operated device could be used for applying antimicrobial steam or for irradiating the artificial turf or crumb rubber with ultraviolet radiation to kill microorganisms on and embedded within the artificial turf or crumb rubber. For example, ultraviolet light from a mercury vapor light (having wavelength of about 254 nm) can be used to kill most microorganisms.

Prior to application of a non-pathogenic microbe that is capable of degrading organic material, the antimicrobial agent can be deactivated to prevent it from killing the non-pathogenic microbe. Deactivation of the ultraviolet irradiation merely requires elimination or termination of the radiation source. Similarly, because ozone dissipates rapidly, deactivation of the ozone antimicrobial agent generally requires terminating the ozone treatment, and waiting an appropriate time to ensure dissipation of the ozone to an Occupational Safety and Health Administration (OSHA) permissible exposure limit (PEL) or other acceptable level. In the cases where a disclosed antimicrobial solution is used, deactivation can be achieved by allowing the artificial turf to dry (or partially dry) or by applying an appropriate neutralizing agent. Mechanical or thermal devices, such as heaters, blowers or vacuums, can be used to reduce drying times.

Because the skin of athletes and others can come in direct contact with the artificial turf, it is desirable to employ antimicrobial agents that do not leave a salt or other residue. For example, a quaternary ammonium antimicrobial agent can be employed, but is less desirable because it can leave a salt residue that might act as an irritant, and which might degrade the appearance of the artificial turf or inhibit a subsequently applied microbial agent. Non-residual antimicrobials that do not leave a residue after application can be used. Examples of non-residual antimicrobials include chlorine dioxide, hydrogen peroxide, ozone, dry steam and ultraviolet radiation.

Non-pathogenic microbes that can be used for digesting or otherwise degrading odor-producing organic materials such as blood, sweat, saliva, urine, feces, foods and beverages, include bacterial and fungal genera such as strains of Bacillus, Lactobacillus, Enterobacter, Streptococcus, Rhizopus, Nitrosomonas, Nitrobacter, Pseudomonas, Alcaligens and Klebsiella. Examples of species of Bacillus include but are not limited to Bacillus megaterium, Bacillus pasteuris, Bacillus laevolactius, Bacillus subtilis, Bacillus licheniformis, Bacillus coagulans, Bacillus pumilus, Bacillus amyloliquefaciens and Bacillus methylotropicus.

The liquid applications of antimicrobial agents and/or subsequent application of liquid containing a beneficial non-pathogenic microbe can be achieved using conventional liquid or foam applicators used on natural turf such as commercially available all-terrain utility sprayers that are widely used for maintaining estates, lawns, pastures, and golf courses. Alternatively, backpack sprays, spray foggers, sprayers attached to a synthetic turf groomer, hand sprayers or the like may be used.

The described embodiments are preferred and/or illustrated, but are not limiting. Various modifications are considered within the purview and scope of the appended claims.

What is claimed is:

1. A process for decontaminating crumb rubber or synthetic turf; with or without infill, comprising steps of:
treating the crumb rubber or synthetic turf, with or without infill, with an antimicrobial agent to kill microorganisms on and embedded within the crumb rubber or synthetic turf;
optionally deactivating the antimicrobial agent; and
applying a non-pathogenic microbe that is capable of degrading organic materials within the crumb rubber or synthetic turf.

2. The process of claim 1, in which the treatment of the crumb rubber or synthetic turf with an antimicrobial agent comprises applying a solution of a peroxide dissolved in water to the crumb rubber or synthetic turf.

3. The process of claim 1, in which the treatment of the crumb rubber or synthetic turf with an antimicrobial agent comprises applying a solution of a hypochlorite dissolved in water to the crumb rubber or synthetic turf.

4. The process of claim 3, in which the hypochlorite is hypochlorous acid, sodium hypochlorite, or calcium hypochlorite.

5. The process of claim 1, in which the treatment of the crumb rubber or synthetic turf with an antimicrobial agent comprises applying a solution of chlorine dioxide dissolved in water to the crumb rubber or synthetic turf.

6. The process of claim 1, in which the treatment of the crumb rubber or synthetic turf with an antimicrobial agent comprises applying a solution of an iodophor dissolved in water to the crumb rubber or synthetic turf.

7. The process of claim 6, in which the iodophor is povidone-iodine or poloxamer iodine complex.

8. The process of claim 6, in which the iodophor is a complex of iodine and a surfactant.

9. The process of claim 2, in which the peroxide is hydrogen peroxide.

10. The process of claim 2, in which the peroxide is a peroxy acid.

11. The process of claim 2, in which the peroxide is an inorganic peroxide.

12. The process of claim 1, in which the treatment of the crumb rubber or synthetic turf with an antimicrobial agent comprises irradiating the crumb rubber or synthetic turf with ultraviolet light at a wavelength effective to kill microorganisms on the crumb rubber or synthetic turf, and deactivating the antimicrobial agent comprises terminating the irradiation of the crumb rubber or synthetic turf.

13. The process of claim 1, in which the treatment of the crumb rubber or synthetic turf with an antimicrobial agent comprises exposing the crumb rubber or synthetic turf to ozone at a concentration effective to kill microorganisms in the crumb rubber or synthetic turf, and deactivating the antimicrobial agent comprises terminating exposure of the crumb rubber or synthetic turf to ozone for a time sufficient to reduce the ozone concentration in the crumb rubber or synthetic turf to an Occupational Safety and Health Administration (OSHA) permissible exposure limit (PEL).

14. The process of claim 1, in which the treatment of the crumb rubber or synthetic turf with an antimicrobial agent comprises exposing the crumb rubber or synthetic turf to chlorine dioxide gas or solution at a concentration effective to kill microorganisms in the crumb rubber or synthetic turf, and deactivating the antimicrobial agent comprises terminating exposure of the crumb rubber or synthetic turf to chlorine dioxide gas for a time sufficient to reduce the chlorine dioxide gas or solution concentration in the crumb rubber or synthetic turf to an Occupational Safety and Health Administration (OSHA) permissible exposure limit (PEL).

15. The process of claim 1, in which the treatment of the crumb rubber or synthetic turf with an antimicrobial agent comprises applying steam to the crumb rubber or synthetic turf.

16. A process in accordance with claim 1, in which the non-pathogenic microbe is of the genus Bacillus.

17. The process of claim 16, in which the genus Bacillus is one or more strains selected from the group of Bacillus megaterium, Bacillus pasteurii, Bacillus laevolacticus, Bacillus pumilus, Bacillus subtilis, Bacillus licheniformis, Bacillus coagulans, Bacillus amyloliquefaciens and Bacillus methylotropicus.

18. A process in accordance with claim 1, in which the non-pathogenic microbe is a bacterial or fungal genera selected from the group consisting of Lactobacillus, Enterobacter, Streptococcus, Rhizopus, Nitrosomonas, Nitrobacter, Pseudomonas, Alcaligens and Klebsiella.

19. A kit for decontaminating crumb rubber or synthetic turf or infill, comprising:
a first container holding powder, liquid or foam comprising an antimicrobial agent;
a second container holding powder or liquid or foam comprising a non-pathogenic microbe that is capable of degrading organic materials; and
instructions for decontaminating crumb rubber, synthetic turf, or infill.

20. The kit of claim 19, in which the non-pathogenic microbe is of the genus Bacillus.

21. The kit of claim 19, in which the non-pathogenic microbe is a bacterial or fungal genera selected from the group consisting of Lactobacillus, Enterobacter, Streptococcus, Rhizopus, Nitrosomonas, Nitrobacter, Pseudomonas, Alcaligens and Klebsiella.

22. A process for sanitizing and/or deodorizing crumb rubber, synthetic turf, or infill comprising:
treating the crumb rubber, synthetic turf or infill with a non-residual antimicrobial agent to kill microorganisms on and embedded within the crumb rubber, synthetic turf, or infill.

23. A process of degrading organic materials on or within crumb rubber, synthetic turf, or infill comprising applying a non-pathogenic microbe that is capable of degrading organic materials on or within the layers of crumb rubber, synthetic turf, or infill.
